SEQUENCE LISTING (3 sequences)

SEQ ID NO: 1 - DNA, 65 bp, Artificial Sequence
  adapter sequence
  misc_feature (34)..(41): n=a,c,g, or t
gatcggaagagcacacgtctgaactccagtcacnnnnnnnnatctcgtatgccgtcttctgcttg

SEQ ID NO: 2 - DNA, 70 bp, Artificial Sequence
  adapter sequence
  misc_feature (30)..(37): n=a,c,g or t
aatgatacggcgaccaccgagatctacacnnnnnnnnacactctttccctacacgacgctcttccgatct

SEQ ID NO: 3 - DNA, 1002 bp, Artificial Sequence
  integration contig sequence of Homo sapiens and Hepatitis B virus
agaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcctccctctctcacttctagggacccttgtggccatatcaggcccaccagataatccaggatgaccttaagatcggctgactggcagccgtgattccacctgcagcctccaccagcctctgccttgcgggtgacacattcacaggttccaggagaaggacgtgggcatctttggggaggggctgtaattgtgcctgccacaagtgcctggggcttctgaaacccaccaaagtttggcaagccccctgcacagcatccttcccaggtgggcacctggcaccaacatcgacggttacagcaggtgcaggaccggcaggagcgtggggctgaggcaggaaaacaaccactccctttcaggggtcctggctggtgtcacccacagcctccacccttgcctgcttctcctccctttctgctttgaactcactcgctccatacacgcttgtctgtggaaggaagctgcttgagatgaagttcaggcctaaggaagtccaaagagct

(19) United States
(12) Patent Application Publication
(10) Pub. No.: US 2021/0207229 A1
(43) Pub. Date: Jul. 8, 2021

(54) HEPATOCELLULAR CARCINOMA SCREENING

(71) Applicant: Seekln, Inc., Lewes, DE (US)

(72) Inventors: Feng Zhang, Brookline, MA (US); Mao Mao, San Diego, CA (US)

(21) Appl. No.: 17/263,345

(22) PCT Filed: Jul. 26, 2019
(86) PCT No.: PCT/US2019/043687

§ 371 Date: Jan. 26, 2021

Related U.S. Application Data
(60) Provisional application No. 62/711,209, filed on Jul. 27, 2018.

(51) Int. Cl. C12Q 1/70; C12Q 1/6886

(52) U.S. Cl. CPC C12Q 1/706; C12Q 2600/156; C12Q 1/6886

(57) ABSTRACT

This disclosure is related to methods for hepatocellular carcinoma (HCC) screening. In one aspect, the disclosure relates to methods of detecting an integration site of hepatitis B vims (HBV) viral DNA in the genome of a subject. The methods involve collecting a nucleic acid sample from the subject; enriching the nucleic acids comprising HIBV sequences in the sample by hybridizing the nucleic acid sample to probes for HIB V viral DNA; sequencing the enriched nucleic acids, thereby obtaining a plurality of sequencing reads; mapping the sequencing reads to both human genome and HBV genome; and detecting the integration site of HIBV viral DNA at the human genome.

### CLAIM OF PRIORITY

This application claims the benefit of U.S. Provisional Application No. 62/711,209, filed on Jul. 27, 2018. The entire contents of the foregoing are incorporated herein by reference.

### TECHNICAL FIELD

This disclosure is related to methods for hepatocellular carcinoma screening.

### BACKGROUND

HBV infection is a major health problem worldwide, especially in developing countries. It is one of the most widespread causes of liver cirrhosis and primary liver cancer (e.g., hepatocellular carcinoma; “HCC”). Chronic HBV infection currently affects millions of people worldwide, and is the main contributor to viral hepatitis-associated morbidity and mortality. The rate is even higher in certain demographic areas.

Early detection of hepatocellular carcinoma can provide better prognosis for patients with HCC. Thus, there is a need to develop screening methods for hepatocellular carcinoma.

### SUMMARY

This disclosure is related to methods for hepatocellular carcinoma screening.

In one aspect, the disclosure relates to methods of detecting an integration site of hepatitis B virus (HBV) viral DNA in the genome of a subject. The methods involve collecting a nucleic acid sample from the subject; enriching the nucleic acids comprising HBV sequences in the sample by hybridizing the nucleic acid sample to probes for HBV viral DNA; sequencing the enriched nucleic acids, thereby obtaining a plurality of sequencing reads; mapping the sequencing reads to both human genome and HBV genome; and detecting the integration site of HBV viral DNA at the human genome.

In some embodiments, the nucleic acid sample is derived from whole blood or plasma of the subject.

In some embodiments, the nucleic acid sample is derived from a tissue sample comprising one or more tumor cells.

In some embodiments, the nucleic acid sample is cell free DNA (cfDNA).

In some embodiments, the nucleic acid sample is circulating tumor DNA (ctDNA).

In some embodiments, the probes for HBV viral DNA are prepared by amplifying HBV genomic DNA.

In some embodiments, the method further comprises: identifying the subject as having hepatocellular carcinoma (HCC) if one or more integration sites for HBV viral DNA in the genome of the subject is detected.

In some embodiments, one or more integration sites are located in one or more loci for oncogenes (e.g., TERT, ABL1 (ABL), ABL2(ABLL,ARG), AKAP13 (HT31, LBC. BRX), ARAF1, ARHGEF5 (TIM), ATF1, AXL, BCL2, BRAF (BRAF1, RAFB1), BRCA1, BRCA2(FANCD1), BRIP1, CBL (CBL2), CSF1R (CSF-1, FMS, MCSF), DAPK1 (DAPK), DEK (D6S231E), DUSP6(MKP3,PYST1), EGF, EGFR (ERBB, ERBB1), ERBB3 (HER3), ERG, ETS1, ETS2, EWSR1 (EWS, ES, PNE,), FES (FPS), FGF4 (HSTF1, KFGF), FGFR1, FGFR10P (FOP), FLCN, FOS (c-fos), FRAP1, FUS (TLS), HRAS, GLI1, GLI2, GPC3, HER2 (ERBB2, TKR1, NEU), HGF (SF), IRF4 (LSIRF, MUM1), JUNB, KIT(SCFR), KRAS2 (RASK2), LCK, LCO, MAP3K8(TPL2, COT, EST), MCF2 (DBL), MDM2, MET(HGFR, RCCP2), MLH type genes, MMD, MOS (MSV), MRAS (RRAS3), MSH type genes, MYB (AMV), MYC, MYCL1 (LMYC), MYCN, NCOA4 (ELE1, ARA70, PTC3), NF1 type genes, NMYC, NRAS, NTRK1 (TRK, TRKA), NUP214 (CAN, D9S46E), OVC, TP53 (P53), PALB2, PAX3 (HUP2) STAT1, PDGFB (SIS), PIM genes, PML (MYL), PMS (PMSL) genes, PPM1D (WIP1), PTEN (MMAC1), PVT1, RAF1 (CRAF), RB1 (RB), RET, RRAS2 (TC21), ROS1 (ROS, MCF3), SMAD type genes, SMARCB1(SNF5, INI1), SMURF1, SRC (AVS), STAT1, STAT3, STATS, TDGF1 (CRGF), TGFBR2, THRA (ERBA, EAR7 etc), TFG (TRKT3), TIF1 (TRIM24, TIF1A), TNC (TN, HXB), TRK, TUSC3, USP6 (TRE2), WNT1 (INT1), WT1, VHL).

In some embodiments, one or more integration sites are located in one or more loci for tumor suppressor genes (e.g., APC, BRCA1, BRCA2(FANCD1), CAPG, CDKN1A (CIP1, WAF1, p21), CDKN2A (CDKN2, MTS1 (depreciated), TP16, p16(INK4)), CD99 (MIC2, MIC2X), FRAP1 (FRAP, MTOR, RAFT1), NF1, NF2, PI5, PDGFRL (PRLTS, PDGRL), PML (MYL), PPARG, PRKAR1A (TSE1), PRSS11 (HTRA, HTRA1)), PTEN (MMAC1), RRAS, RB1 (RB), SEMA3B, SMAD2 (MADH2, MADR2), SMAD3 (MADH3), SMAD4 (MADH4, DPC4), SMARCB1 (SNF5, INI1), ST3 (TSHL, CCTS), TET2, TOP1, TNC (TN, HXB), TP53 (P53), TP63 (TP73L), TP73, TSG11, TUSC2 (FUS1), TUSC3, VHL).

In some embodiments, one or more integration sites are located in one or more loci for cancer-associated genes (e.g., CD55, ICAM, MCAM, and ALCAM).

In some embodiments, one or more integration sites are located in one or more genes selected from the group consisting of TERT, MLL4, CCNE1, SENP5, ROCK1, FN1, PTPRD, UNC5D, NRG3, CTNND2 and AHRR.

In some embodiments, the method further comprising: identifying the subject as having hepatocellular carcinoma (HCC) if the total number of the integration sites is over a reference threshold (e.g., 1, 2, 3, 4, 5, 6, 7, 8, 9, 10, 11, 12, 13, 14, 15, 16, 17, 18, 19, 20, 30, 31, 32, 33, 34, 35, 36, 37, 38, 39, 40, 50, 60, 70, 80, 90, 100, 110, 120, 130, 140, 150, 160, 170, 180, 190, or 200).

In some embodiments, the subject has hepatitis B. In some embodiments, the method further comprises treating HCC in the subject.

In one aspect, the disclosure provides a method of detecting an integration site of hepatitis B virus (HBV) viral DNA in the genome of a subject. The method involves one or more of the following steps: collecting a nucleic acid sample from the subject; sequencing the nucleic acid sample by paired end sequencing, thereby obtaining a plurality of paired end sequencing reads; identifying one or more paired end sequencing reads that are mapped to a HBV integration site, wherein (1) one end of the paired end sequencing reads is mapped to HBV viral DNA, and the other end of the paired end sequencing reads is mapped to human genome; or (2) one end of the paired end sequencing reads comprises a sequence that is mapped to HBV viral DNA, and a sequence that is mapped to human genome; detecting the integration site of HBV viral DNA in the subject.

In some embodiments, the method further comprises prior to sequencing the nucleic acid sample by paired end sequencing, enriching nucleic acids comprising HBV sequences in the sample by hybridizing the nucleic acid sample to probes for HBV viral DNA.

In some embodiments, the integration site of HBV viral DNA has more than three paired end sequencing reads that are mapped to the HBV integration site.

In some embodiments, the method further comprises constructing a HBV integration site sequence based on one or more paired end sequencing reads that are mapped to the HBV integration site; and aligning one or more paired end sequencing reads to the constructed HBV integration site sequence.

In some embodiments, the method further comprises determining one or more HBV integration sites are located in one or more genes selected from the group consisting of TERT, MLL4, CCNE1, SENP5, ROCK1, FN1, PTPRD, UNC5D, NRG3, CTNND2 and AHRR; and determining that the subject has HCC.

In some embodiments, the method further comprises: determining a probability that the subject has HCC based on one or more of the following: (1) total number of paired end sequencing reads in the subject, each having one end that is mapped to HBV viral DNA, and one end that is mapped to human genome; (2) total number of paired end sequencing reads in the subject, each having one end that comprises a sequence that is mapped to HBV viral DNA, and a sequence that is mapped to human genome; and (3) total number of HBV integration sites in the subject.

In some embodiments, the probability is calculated based on the following equation:

$$P = \frac{1}{1 + {e}^{- ( α + {β}_{1} * {x}_{1} + {β}_{2} * {x}_{2} + {β}_{3} * {x}_{3} )}}$$

In some embodiments, X₁ is the total number of paired end sequencing reads in the subject, each having one end that is mapped to HBV viral DNA, and one end that is mapped to human genome; X₂ is the total number of paired end sequencing reads in the subject, each having one end that comprises a sequence that is mapped to HBV viral DNA, and a sequence that is mapped to human genome; X₃ is the total number of HBV integration sites in the subject; and α is a constant, β₁, β₂, and β₃ are coefficients of a logistic regression.

In some embodiments, the subject has hepatitis B.

In one aspect, the disclosure provides a method of screening a subject for hepatocellular carcinoma (HCC), the method comprising one or more of the following steps: collecting a nucleic acid sample from the subject; sequencing the nucleic acid sample, thereby obtaining a plurality of sequencing reads; mapping the sequencing reads to both human genome and HBV genome; and detecting one or more integration sites of HBV viral DNA in the subject's genome, thereby determining that the subject has HCC.

In some embodiments, the method further comprises enriching nucleic acids comprising HBV viral DNA sequences in the nucleic acid sample by hybridizing the nucleic acid sample to probes for HBV viral DNA.

In some embodiments, the nucleic acid sample is sequenced by paired end sequencing. In some embodiments, the subject has hepatitis B.

In some embodiments, the nucleic acid sample comprises cfDNA. In some embodiments, the method further comprises performing biopsy or imaging on the subject.

In some embodiments, the method further comprises treating HCC in the subject. In some embodiments, the subject is treated by surgery, chemotherapy, or immunotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing methods of performing hepatocellular carcinoma (HCC) screening.

FIG. 2A is a schematic diagram showing the paired end (PE) supporting reads that are mapped to both human genome and HVB genome. ReadA_1 and readA_2 are paired end sequence reads and are derived from the same cfDNA molecule. One read is mapped to the human genome, and the other read is mapped to the HBV genome, indicating this cfDNA molecule has an integration site.

FIG. 2B is a schematic diagram showing HBV and human genome splicing supporting reads. At least one read of the paired end sequences is mapped to both the human genome and the HBV genome. This indicates that the integration site is in one of these paired end sequence. The other end can be mapped to the HBV genome (see e.g., readB_2), the human genome (see e.g., readC_1), or can contain the same integration site (see e.g., readA_2).

FIG. 3 is a schematic diagram showing re-mapping paired end sequences to HBV integration contig sequence. In some cases, only a part of reads can be mapped to the human or HBV genome (see e.g., the solid line of readA/readB), and the other part of reads cannot be properly mapped (the dotted line of readA/readB). Once the HBV integration contig sequence is constructed, the unmapped sequences can be successfully mapped to the HBV integration contig sequence.

FIG. 4 is a graph showing one HBV integration site in a subject. The first 500 bp of integration contig is from the HBV genome, the next 500 bp is from human genome chromosome 5. The upper panel shows the coverage around the integration site. The lower panel shows alignment of the supporting reads to this integration site.

FIG. 5 is a graph showing the fragment lengths of DNA molecules in one DNA library constructed by plasma sample.

### DETAILED DESCRIPTION

This disclosure is related to methods for hepatocellular carcinoma screening. HBV DNA integration into host genome is a compelling step during chronic hepatitis B infection. HBV integration in human genome is a unique and specific event of HBV-related HCC. The present disclosure provides screening methods for subjects having hepatocellular carcinoma, and methods of enriching HBV integration site sequences from human plasma DNA (e.g., cell free DNA).

### Hepatocellular Carcinoma

Hepatocellular carcinoma (HCC) is the most common type of primary liver cancer in adults. It often occurs in patients with chronic liver inflammation, and it is closely linked to chronic viral hepatitis infection (e.g., hepatitis B). Certain diseases, such as hemochromatosis and alpha 1-antitrypsin deficiency, markedly increase the risk of developing HCC. Metabolic syndrome and nonalcoholic steatohepatitis (NASH) are also increasingly recognized as risk factors for HCC. The vast majority of HCC occurs in Asia and sub-Saharan Africa, where hepatitis B infection is endemic.

HCC remains associated with a high mortality rate, in part related to initial diagnosis commonly at an advanced stage of disease. As with other cancers, outcomes are significantly improved if treatment is initiated earlier in the disease process. Because the vast majority of HCC occurs in people with certain chronic liver diseases, especially those with cirrhosis, liver screening is commonly recommended for this population. The present disclosure provides methods of screening a subject for HCC. Once the HCC is confirmed, the treatment can be initiated when HCC is still in the early stage.

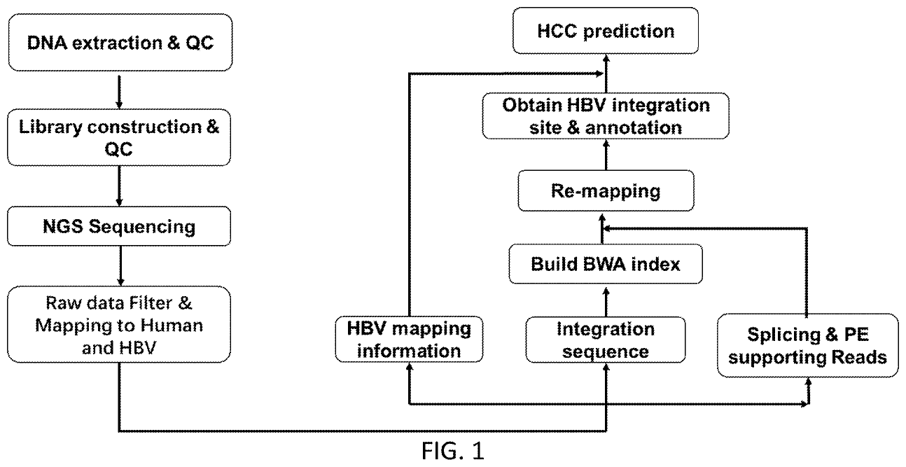

FIG. 1 shows an exemplary procedure of performing hepatocellular carcinoma (HCC) screening. In some embodiments, cell free DNAs are extracted from the subject. The library for sequencing can be prepared. In some embodiments, the sequences are further enriched for HBV sequences. Next generation sequencing (e.g., paired-end sequencing) can be performed. The sequence results can be used to detect HBV integration sites, thereby determining whether the subject has HCC.

In some embodiments, if the screening method as described herein determines that the subject has HCC, or is likely to have HCC, further medical procedures are then performed to confirm that the subject has HCC (e.g., biopsy or imaging). Usually, a biopsy of the tumor is often required to prove the diagnosis. However, imaging can also be used to confirm the diagnosis. These imaging techniques include e.g., ultrasound, CT scan, and MRI.

In some embodiments, if further medical procedures cannot confirm that the subject has HCC, further monitoring will be performed. For example, the methods described herein including e.g., sequencing and imaging, can be performed every 1, 2, 3, 4, 5, 6 months, every year, or every two years. In some embodiments, blood levels of tumor marker alpha-fetoprotein (AFP) are measured. In some embodiments, life style changes are recommended to the subject (e.g., reducing alcohol intake).

In some embodiments, if the subject is confirmed to have HCC, an appropriate treatment can be administered to the subject. Treatment of hepatocellular carcinoma varies by the stage of disease, a person's likelihood to tolerate surgery, and availability of liver transplant. Some common treatment for hepatocellular carcinoma includes e.g., surgery, liver transplant surgery, radiofrequency ablation, cryoablation, ablation using alcohol or microwaves, chemotherapy, radiation, targeted drug therapy, and immunotherapy etc. For limited cases, surgically removing the malignant cells can be curative. This may be accomplished by resection of the affected portion of the liver (partial hepatectomy) or in some cases by orthotopic liver transplantation of the entire organ.

### Sample Preparation

The present disclosure provides a fast, accurate, and cost-effective way to screen HCC in a subject. As used herein, the terms “subject” and “patient” are used interchangeably throughout the specification and describe an animal, human or non-human, to whom the methods as described herein is provided. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject has or is suspected to have HCC. In some embodiments, the subject is at risk of developing HCC. For example, the subject has chronic viral hepatitis infection (e.g., hepatitis B or C), hemochromatosis and alpha 1-antitrypsin deficiency, metabolic syndrome, and/or nonalcoholic steatohepatitis (NASH). In some embodiments, the subject has an elevated level of tumor marker alpha-fetoprotein (e.g., as compared to a reference threshold). In some embodiments, the subject has hepatitis B or has a history of hepatitis B infection.

Nucleic acid samples can be collected from a subject or a group of subjects. Provided herein are methods and compositions for analyzing nucleic acids (e.g., for screening hepatocellular carcinoma). In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, cell or tissue origins, tumor origins, cancer origins, sample origins, subject origins, fetal origins, maternal origins), or combinations thereof.

Nucleic acid samples can be isolated from any type of suitable biological specimen or sample (e.g., a test sample). A sample or test sample can be any specimen that is isolated or obtained from a subject (e.g., a human subject). Non-limiting examples of specimens include fluid or tissue from a subject, including, without limitation, blood, serum, umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., tumor cells, and liver tissue), celocentesis sample, fetal cellular remnants, urine, feces, sputum, saliva, nasal mucous, prostate fluid, lavage, semen, lymphatic fluid, bile, tears, sweat, breast milk, breast fluid, embryonic cells, fetal cells (e.g. placental cells).

In some embodiments, a biological sample can be blood, plasma or serum. As used herein, the term “blood” encompasses whole blood or any fractions of blood, such as serum and plasma. Blood or fractions thereof can comprise cell-free or intracellular nucleic acids. Blood can comprise buffy coats. Buffy coats are sometimes isolated by utilizing a ficoll gradient. Buffy coats can comprise white blood cells (e.g., leukocytes, T-cells, B-cells, platelets). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted can be acellular (e.g., cell-free). In some embodiments, a fluid or tissue sample can contain cellular elements or cellular remnants. In some embodiments, cancer cells or tumor cells can be included in the sample.

A sample often is heterogeneous. In many cases, more than one type of nucleic acid species is present in the sample. For example, heterogeneous nucleic acid can include, but is not limited to, cancer and non-cancer nucleic acid, pathogen and host nucleic acid, and/or mutated and wild-type nucleic acid. A sample may be heterogeneous because more than one cell type is present, such as a cancer and non-cancer cell, or a pathogenic and host cell.

In some embodiments, the sample comprise cell free DNA (ctDNA) or circulating tumor DNA (ctDNA). As used herein, the term “cell-free DNA” or “cfDNA” refers to DNA that is freely circulating in the bloodstream. These ctDNA can be isolated from a source having substantially no cells. In some embodiments, these extracellular nucleic acids can be present in and obtained from blood. Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample). Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species. For example, blood serum or plasma from a person having cancer can include nucleic acid from cancer cells and nucleic acid from non-cancer cells. As used herein, the term “circulating tumor DNA” or “ctDNA” refers to tumor-derived fragmented DNA in the bloodstream that is not associated with cells. ctDNA usually originates directly from the tumor or from circulating tumor cells (CTCs). The circulating tumor cells are viable, intact tumor cells that shed from primary tumors and enter the bloodstream or lymphatic system. The ctDNA can be released from tumor cells by apoptosis and necrosis (e.g., from dying cells), or active release from viable tumor cells (e.g., secretion). In some embodiments, the length of ctDNA or cfDNA can be at least or about 160, 170, 180, 190, 200, 210, 220, 230, 240, 250, 260, 270, 280, 290, 300, 310, 320, 330, 340, 350, 360, 370, 380, 390, or 400 bp. In some embodiments, the length of ctDNA or cfDNA can be less than about 160, 170, 180, 190, 200, 210, 220, 230, 240, 250, 260, 270, 280, 290, 300, 310, 320, 330, 340, 350, 360, 370, 380, 390, or 400 bp.

The present disclosure provides methods of separating, enriching and analyzing cell free DNA or circulating tumor DNA found in blood as a non-invasive means to detect the presence and/or to monitor the progress of a cancer (e.g., HCC). Thus, the first steps of practicing the methods described herein are to obtain a blood sample from a subject and extract DNA from the subject.

A blood sample can be obtained from a subject (e.g., a subject who is suspected to have HCC or at risk of developing HCC). The procedure can be performed in hospitals or clinics. An appropriate amount of peripheral blood, e.g., typically between 1 and 50 ml (e.g., between 1 and 10 ml), can be collected. Blood samples can be collected, stored or transported in a manner known to the person of ordinary skill in the art to minimize degradation or the quality of nucleic acid present in the sample. In some embodiments, the blood can be placed in a tube containing EDTA to prevent blood clotting, and plasma can then be obtained from whole blood through centrifugation. Serum can be obtained with or without centrifugation-following blood clotting. If centrifugation is used then it is typically, though not exclusively, conducted at an appropriate speed, e.g., 1,500-3,000×g. Plasma or serum can be subjected to additional centrifugation steps before being transferred to a fresh tube for DNA extraction. In some embodiments, the samples can be centrifuged at about 1600 g. In some embodiments, the samples are processed within 2 hours of collection. In some embodiments, the supernatants is further centrifuged at 16,000 g for 10 min at 4° C., and plasma is harvested and can be stored at −80° C. until further use. In some embodiments, cfDNA population can be maintained by inhibiting nuclease activity and stabilizing white blood cells in the blood collection tube. In these cases, the samples can be stored for up to 14 days at temperatures between 6° C. and 37° C. Some of these methods are described e.g., in Diaz et al. “Performance of Streck cfDNA blood collection tubes for liquid biopsy testing.” PLoS One 11.11 (2016): e0166354, which is incorporated herein by reference in its entirety.

There are numerous known methods for extracting DNA from a biological sample including blood. The general methods of DNA preparation (e.g., described by Sambrook and Russell, Molecular Cloning: A Laboratory Manual 3d ed., 2001) can be followed; various commercially available reagents or kits, such as Qiagen's QIAamp Circulating Nucleic Acid Kit, QIAamp DNA Mini Kit or QIAamp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample. In some embodiments, cell free (cfDNA) can be extracted from plasma using appropriate kits (e.g., the QIAamp Circulating Nucleic Acid kit (QIAGEN)). In some embodiments, DNA can be quantified with the Qubit Fluorometer and the Qubit dsDNA HS Assay kit (Life Technologies, Carlsbad, Calif.).

cfDNA purification is prone to contamination due to ruptured blood cells during the purification process. Because of this, different purification methods can lead to significantly different cfDNA extraction yields. In some embodiments, purification methods involve collection of blood via venipuncture, centrifugation to pellet the cells, and extraction of cfDNA from the plasma. In some embodiments, after extraction, cell-free DNA can be about or at least 50% of the overall nucleic acid (e.g., about or at least 50%, 60%, 70%, 80%, 90%, 91%, 92%, 93%, 94%, 95%, 96%, 97%, 98%, or 99% of the total nucleic acid is cell-free DNA).

The nucleic acid that can be analyzed by the methods described herein include, but are not limited to, DNA (e.g., complementary DNA (cDNA), genomic DNA (gDNA), cfDNA, or ctDNA), ribonucleic acid (RNA) (e.g., message RNA (mRNA), short inhibitory RNA (siRNA), ribosomal RNA (rRNA), transfer RNA (tRNA), or microRNA), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. A nucleic acid can be in any form useful for conducting processes herein (e.g., linear, circular, supercoiled, single-stranded, or double-stranded). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures.

In some embodiments, the nucleic acid can be extracted, isolated, purified, partially purified or amplified from the samples before sequencing. In some embodiments, nucleic acid can be processed by subjecting nucleic acid to a method that generates nucleic acid fragments. Fragments can be generated by a suitable method known in the art, and the average, mean or nominal length of nucleic acid fragments can be controlled by selecting an appropriate fragment-generating procedure.

### Library Construction and HBV Sequence Enrichment

The library can be prepared for nucleic acid samples (e.g., cfDNA). In some embodiments, the End Repair and 3′-end dA-tailing are performed. End repair is performed to ensure that DNA molecules are free of overhangs. Then T-tailed adapters and a 3′dA overhang is enzymatically added to the DNA molecules. The reaction products can be cleaned (e.g., by magnetic beads) and amplified.

Library purity and concentration can be quantified (e.g., by Qubit Fluorometer and the Qubit dsDNA HS Assay kit). Fragment length can be determined (e.g., on a Bioanalyzer using the DNA 1000 Kit).

In some embodiments, multiplexed libraries are used. Multiplex sequencing allows large numbers of libraries to be pooled and sequenced simultaneously during a single run on a high-throughput instrument. Individual “barcode” sequences can be added to each DNA fragment during next-generation sequencing (NGS) library preparation. Nucleic acid samples from different subjects can be pooled together. Thus, in some embodiments, the library can contain nucleic acids from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

HBV probes can be generated by HBV virus genomes or obtained commercially. In some embodiments, HBV genomic DNA can be extracted from clinical serum samples. Full-length HBV virus genome can be amplified by PCR. Amplicons are purified and then fragmented. In some embodiments, fragments with appropriate size (e.g., about 100 bp to 150 bp) are selected. In some embodiments, single-stranded HBV probes can be generated by high temperature denaturation (e.g., at 94° C. for 5 min). In some embodiments, these HBV probes are labeled by biotin. In some embodiments, prior to next-generation sequencing (NGS), HBV probes are hybridized to a sequencing library in solution. The biotinylated probe/target hybrids are pulled down by streptavidin-coated magnetic beads to obtain libraries highly enriched for the target regions.

In some embodiments, libraries are hybridized with HBV probes (e.g., for 16˜24 hours) and then are washed to remove un-captured fragments. In some embodiments, the captured DNA fragments are amplified following hybrid selection (e.g., by about 12˜15 cycles of PCR). The reaction products can be purified by magnetic beads (e.g., Agencourt® AMPure XP beads).

### Sequencing

Nucleic acids (e.g., nucleic acid fragments, sample nucleic acid, cell-free nucleic acid, circulating tumor nucleic acids) are sequenced before the analysis. As used herein, “reads” or “sequence reads” are short nucleotide sequences produced by any sequencing process described herein or known in the art. Reads can be generated from one end of nucleic acid fragments (e.g., single-end reads), and sometimes are generated from both ends of nucleic acids (e.g., paired-end reads).

Sequence reads obtained from cell-free DNA can be reads from a mixture of nucleic acids derived from normal cells or tumor cells. A mixture of relatively short reads can be transformed by processes described herein into a representation of a genomic nucleic acid present in a subject.

Sequence reads can be mapped and the number of reads or sequence tags mapping to a specified nucleic acid region (e.g., a chromosome, a bin, a genomic section) are referred to as counts. In some embodiments, counts can be manipulated or transformed (e.g., normalized, combined, added, filtered, selected, averaged, derived as a mean, the like, or a combination thereof).

In some embodiments, a group of nucleic acid samples from one individual are sequenced. In certain embodiments, nucleic acid samples from two or more samples, wherein each sample is from one individual or two or more individuals, are pooled and the pool is sequenced together. In some embodiments, a nucleic acid sample from each biological sample often is identified by one or more unique identification tags.

The nucleic acids can also be sequenced with redundancy. A given region of the genome or a region of the cell-free DNA can be covered by two or more reads or overlapping reads (e.g., “fold” coverage greater than 1). Coverage (or depth) in DNA sequencing refers to the number of unique reads that include a given nucleotide in the reconstructed sequence. In some embodiments, the fold is calculated based on the reference sequence (e.g., HBV genome).

In some embodiments, the nucleic acid is sequenced with about 1-fold to about 1000-fold coverage. In some embodiments, sequencing is performed by about or at least 1, 2, 3, 4, 5, 6, 7, 8, 9, 10, 15, 20, 30, 40, 50, 60, 70, 80, 90, 100, 200, 300, 400, 500, or 1000 fold coverage. In some embodiments, sequencing is performed by no more than 10, 15, 20, 30, 40, 50, 60, 70, 80, 90, 100, 200, 300, 400, 500, or 1000 coverage.

In some embodiments, a sequencing library can be prepared prior to or during a sequencing process. Methods for preparing the sequencing library are known in the art and commercially available platforms may be used for certain applications. Certain commercially available library platforms may be compatible with sequencing processes described herein. For example, one or more commercially available library platforms may be compatible with a sequencing by synthesis process.

Any sequencing method suitable for conducting methods described herein can be used. In some embodiments, a high-throughput sequencing method is used. High-throughput sequencing methods generally involve clonally amplified DNA templates or single DNA molecules that are sequenced in a massively parallel fashion within a flow cell. Such sequencing methods also can provide digital quantitative information, where each sequence read is a countable “sequence tag” or “count” representing an individual clonal DNA template, a single DNA molecule, bin or chromosome.

Next generation sequencing techniques capable of sequencing DNA in a massively parallel fashion are collectively referred to herein as “massively parallel sequencing” (MPS). High-throughput sequencing technologies include, for example, sequencing-by-synthesis with reversible dye terminators, sequencing by oligonucleotide probe ligation, pyrosequencing and real time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single molecule sequencing, single molecule real time (SMRT) sequencing, nanopore sequencing, ION Torrent and RNA polymerase (RNAP) sequencing. Some of these sequencing methods are described e.g., in US20130288244A1, which is incorporated herein by reference in its entirety.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, paired end (PE) sequencing is performed. Paired-end sequencing provides sequences of both ends of a fragment. PE sequencing involves sequencing both ends of the DNA fragments in a library and aligning the forward and reverse reads as read pairs. In addition to producing twice the number of reads for the same time and effort in library preparation, sequences aligned as read pairs enable more accurate read alignment and the ability to detect indels. Analysis of differential read-pair spacing also allows removal of PCR duplicates, a common artifact resulting from PCR amplification during library preparation. In some embodiments, the sequence between the two ends of a fragment cannot be sequenced. In some embodiments, the sequence from both ends can cover the entire sequence of the fragment. In some embodiments, the libraries can be sequenced by flow cell-based sequencing instrument (e.g., using 150 bp paired-end runs on an IlluminaHiseq Xten).

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g., about or at least 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp). In some embodiments, the sequence reads are of a mean, median or average length of about 1000 bp or more. In some embodiments, the sequence reads are of less than 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp are removed because of poor quality.

Mapping nucleotide sequence reads (i.e., sequence information from a fragment whose physical genomic position is unknown) can be performed in a number of ways, and often comprises alignment of the obtained sequence reads with a matching sequence in a reference genome (e.g., Li et al., “Mapping short DNA sequencing reads and calling variants using mapping quality score,” Genome Res., 2008 Aug. 19.) In such alignments, sequence reads generally are aligned to a reference sequence and those that align are designated as being “mapped” or a “sequence tag.” In certain embodiments, a mapped sequence read is referred to as a “hit” or a “count”.

As used herein, the terms “aligned”, “alignment”, or “aligning” refer to two or more nucleic acid sequences that can be identified as a match (e.g., 100% identity) or partial match. Alignments can be done manually or by a computer algorithm, examples including the Efficient Local Alignment of Nucleotide Data (ELAND) computer program distributed as part of the Illumina Genomics Analysis pipeline. The alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, or 50% match. In some embodiments, an alignment comprises a mismatch. In some embodiments, an alignment comprises 1, 2, 3, 4 or 5 mismatches. Two or more sequences can be aligned using either strand. In certain embodiments, a nucleic acid sequence is aligned with the reverse complement of another nucleic acid sequence.

Various computational methods can be used to map each sequence read to a genomic region. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. In some embodiments, sequence reads can be aligned with sequences in a reference genome (e.g., human genome or/and HBV genome). In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate genomic sections, for example. Some of the methods of analyzing sequence reads are described e.g., US20130288244A1, which is incorporated herein by reference in its entirety.

### Analyzing Sequencing Data

### Raw Data Cleaning

In order to increase sequencing data quality, filtering can be performed. In some embodiments, data cleaning can include one or more of the following: (1) removing reads containing sequencing adapter or cutting adapter sequence from reads containing sequencing adapter; (2) removing reads whose low-quality base ratio is more than a pre-determined threshold (e.g., 50%); (3) removing reads whose undetermined base (‘N’ base) ratio is more than a pre-determined threshold (e.g., 5%). Statistical analysis of data and downstream bioinformatics analysis can also be performed to clean the sequencing data.

### Initial Mapping

After data cleaning, sequence reads are mapped to human genome and HBV genome. Those pair-end reads that are only mapped to human are removed, because these sequence reads do not have integration site information.

### HBV Mapping

In some embodiments, sequencing reads which are partially aligned to human genome and partially aligned to HBV genome are selected. After filtering low mapping quality reads, reads are mapped to the HBV genome from the bam file. These reads include:

“HBV mapping reads”: both of the paired end reads are mapped to the HBV genome. HBV mapping reads represent the virus content in the patient sample.

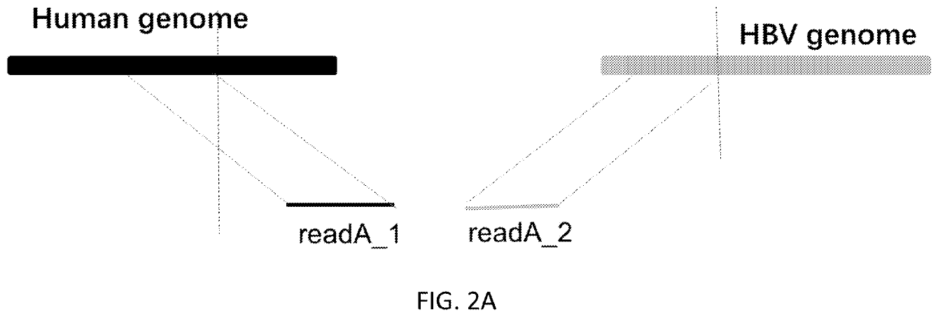

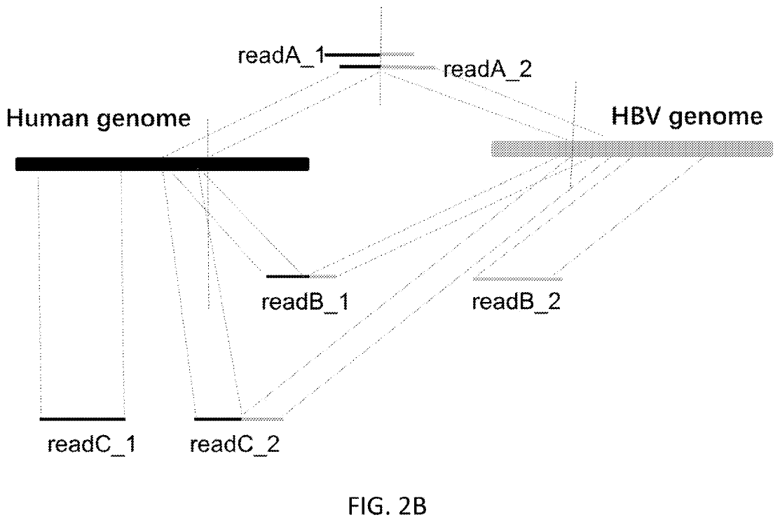

“PE supporting reads”: One read of the paired end reads is mapped to the human genome and the other paired end read is mapped to the HBV genome (FIG. 2A).

“Splicing supporting reads”: the integration site is located on at least one paired end read. Thus, a part of that paired end read is mapped to the human and a part of the same paired end read is mapped to the HBV genome (FIG. 2B).

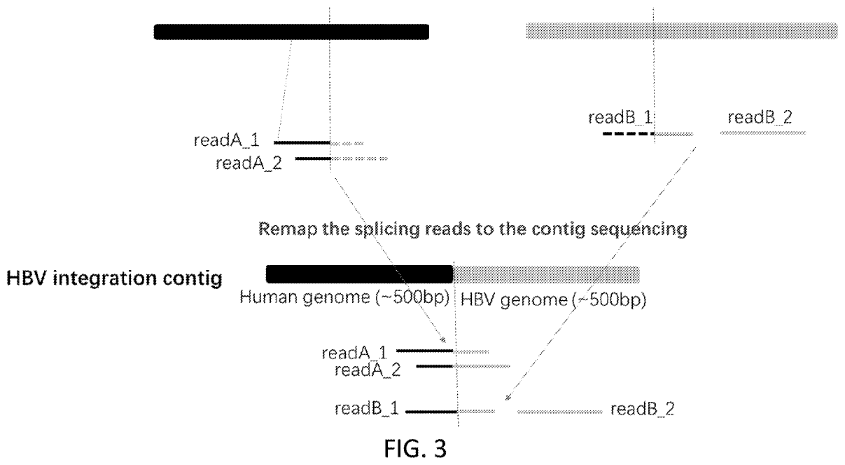

In some embodiments, splicing supporting reads are extracted (FIG. 3). The fastq file can be re-constructed based on the previous extracting reads except HBV mapping reads.

### Construct the Contig Sequence Based on the Splicing Reads

As shown in FIG. 2B, the integration sites and the breakpoints can be identified from the splicing read sequences. The HBV integration site in the human genome can be determined. Then the ‘fasta’ sequence (e.g., 100 bp-1000 bp) around the breakpoints from human/HBV genome can be extracted, the human and HBV ‘fasta’ sequence can be joined as integrating contig sequence. The index can be rebuilt by e.g., BWA software.

### Re-Mapping

BWA re-indexed “fasta” file can be used as “reference genome” with candidate integration contig sequencing. Re-constructed “fastq” file can be aligned to the “reference genome” file. Based on the re-mapping bam file, reads are mapped to the integrating contig.

If the number of supporting reads is not greater than a predetermined threshold (e.g., 1, 2, 3, 4, or 5), the integration contig is filtered.

In some embodiments, the integration contig sequences are then annotated by the human genome. In some embodiments, to determine the HBV integration breakpoints, if the length of sequencing reads is short (such as PE50, PE75, PE90, PE100), the PE-assembled contigs are also used, and are re-mapped to human and HBV genome reference respectively using BWA. In some embodiments, reserved contigs can have a match length larger than 30 bp both on HBV genome reference and human genome reference. The reserved PE-assembled reads can be used to detect integration sites and breakpoints. The joint position of human and HBV sequence are the breakpoints for HBV integration.

### HCC Prediction

In some embodiments, the individual is determined to be a HCC patient or is determined to be likely to have HCC if one or more HBV integration sites are detected by sequencing in the individual's plasma DNA.

In some embodiments, if the HBV integration site is confirmed (e.g., detected with high confidence), the individual is determined to be a HCC patient or is determined to be likely to have HCC. The HBV integration site is detected with high confidence when the number of splicing supporting reads and/or the PE supporting reads that are mapped to the same integration site is more than a predetermined threshold. In some embodiments, the predetermined threshold is 2, 3, 4, 5, 6, 7, 8, 9, or 10. In some embodiments, the predetermined threshold is 3.

In some embodiments, the HBV integration site cannot be confirmed with high confidence (e.g., with at least 3 splicing supporting reads and/or the PE supporting reads that are mapped to the same integration site). But if the number of unique splicing supporting reads or the unique PE supporting reads is more a predetermined threshold, the subject can be determined as having an increased risk of developing HCC. In some embodiments, further monitoring and testing is required. In some embodiments, if the number of unique splicing supporting reads or the unique PE supporting reads is less than a predetermined threshold, the subject can be determined as not having HCC.

In some embodiments, logistic regression is performed and applied to a dataset that includes a group of patients with HCC, and a group of patients without HCC. In some embodiments, all patients in the dataset have hepatitis B or a history of HBV infection.

A logistic regression model is a non-linear transformation of the linear regression. The logistic regression model is often referred to as the “logit” model and can be expressed as

ln [p/(1−p)]=α+β₁X₁+β₂X₂+ . . . +βₖXₖ+ε
where,
α and ε are constants
ln is the natural logarithm, log₍ₑ₎, where e=2.71828 . . . ,
p is the probability that the event Y occurs, p(Y=1),
p/(1−p) is the “odds ratio,”
ln [p/(1−p)] is the log odds ratio, or “logit”.

It will be appreciated by those of skill in the art that a and c can be folded into a single constant, and expressed as a. In some embodiments, a single term a is used, and is omitted. The “logistic” distribution is an S-shaped distribution function. The logit distribution constrains the estimated probabilities (p) to lie between 0 and 1.

In some embodiments, the logistic regression model is expressed as

Y=α+ΣβᵢXᵢ

Here, Y is a value indicating a probability that the set of predictor levels classifies with the set of levels for subjects with HCC, as opposed to the set of levels for subjects without HCC. In some embodiments, the set of predictor levels include e.g., the total number of unique PE supporting reads, the total number of unique splicing supporting reads, and the number of confirmed integration sites in a subject.

In some embodiments, X1 can be the number of unique PE supporting reads, X2 can be the number of unique splicing supporting reads, and X3 can be the number of confirmed integration sites or confirmed integration events. βi is a logistic regression equation coefficient for the predictor, α is a logistic regression equation constant that can be zero, and βi and α are the result of applying logistic regression analysis to the set of levels for subjects with HCC and the set of levels for subjects without HCC.

In some embodiments, the logistic regression model is fit by maximum likelihood estimation (MLE). The coefficients (e.g., α, β1, β2, . . . ) are determined by maximum likelihood. A likelihood is a conditional probability (e.g., P(Y|X), the probability of Y given X). The likelihood function (L) measures the probability of observing the particular set of dependent variable values (Y1, Y2, . . . , Yn) that occur in the sample data set. In some embodiments, it is written as the product of the probability of observing Y1, Y2, . . . , Yn:

L=Prob(Y1,Y2, . . . ,Yn)=Prob(Y1)*Prob(Y2)* . . . Prob(Yn)

The higher the likelihood function, the higher the probability of observing the Ys in the sample. MLE involves finding the coefficients (α, β1, β2, . . . ) that make the log of the likelihood function (LL<0) as large as possible or −2 times the log of the likelihood function (−2LL) as small as possible. In MLE, some initial estimates of the parameters α, β1, β2, and so forth are made. Then, the likelihood of the data given these parameter estimates is computed. The parameter estimates are improved, and the likelihood of the data is recalculated. This process is repeated until the parameter estimates remain substantially unchanged (for example, a change of less than 0.01 or 0.001). Examples of logistic regression and fitting logistic regression models are found in Hastie, The Elements of Statistical Learning, Springer, N.Y., 2001, pp. 95-100.

Once the logistic regression equation coefficients and the logistic regression equation constant are determined, the classifier can be readily applied to a test subject to obtain Y. In one embodiment, Y can be used to calculate probability (p) by solving the function Y=In (p/(1−p)).

In some embodiments, the probability that a subject has HCC can calculated based on the following equation:

$$P ( H C C ) = \frac{1}{1 + {e}^{- ( α + Σ {β}_{i} * {x}_{i} )}}$$

Wherein X₁ is the number of unique PE supporting reads, X2 is the number of unique splicing supporting reads, and X3 is the number of confirmed integration events.

In some embodiments, if the HBV integration site is located at one or more HCC-related HBV integration genes, the subject is predicted to be a HCC patient. In some embodiments, the one or more HCC-related HBV integration genes are selected from TERT, MLL4, CCNE1, SENP5, ROCK1, FN1, PTPRD, UNC5D, NRG3, CTNND2, and AHRR. In some embodiments, the HBV integration site located at one or more HCC-related HBV integration genes is detected with high confidence. If the subject does not have any confirmed HBV integration sites at HCC-related HBV integration genes, then the probability that the subject has HCC can be calculated. In some embodiments, if the probability is higher than a pre-determined threshold, the subject is predicted to have HCC; otherwise, the subject is predicted not to have HCC.

In some embodiments, the methods as described herein can properly determine whether a subject has HCC. The methods can be evaluated by sensitivity and specificity. In one embodiment, a Receiver Operating Characteristic (ROC) is used to evaluate the methods as described herein. The ROC provides several parameters to evaluate both the sensitivity and the specificity of the result of the equation generated. In one embodiment, the ROC area (area under the curve) can be used. A ROC area greater than 0.5, 0.6, 0.7, 0.8, or 0.9 is preferred. A perfect ROC area score of 1.0 is indicative of both 100% sensitivity and 100% specificity. In some embodiments, the sensitivity can be greater than 0.95, 0.9, 0.85, 0.8, 0.7, 0.65, 0.6, 0.55, or 0.5. In some embodiments, the specificity can be greater than 0.95, 0.9, 0.85, 0.8, 0.7, 0.65, 0.6, 0.55, or 0.5.

The present disclosure also provides methods of monitoring the progress of a cancer (e.g., HCC). In some embodiments, an increase of the number of HBV integration sites in the subject indicates that HCC is progressing to a higher stage. Similarly, an increase of the probability as described herein can indicate that HCC is progressing to a higher stage. In some embodiments, the subject is treated by a treatment for HCC. Thus, in some embodiments, a decrease of the number of HBV integration sites in the subject or a decrease of the probability as described herein can indicate that the treatment is effective.

### Methods of Treatment

The present disclosure provides methods of treating cancer (e.g., liver cancer, HCC). In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject. In some embodiments, the methods described herein can be used to monitor or track the effectiveness of the treatments.

The treatments can generally include e.g., surgery, chemotherapy, radiation therapy, hormonal therapy, immunotherapy, targeted therapy, and/or a combination thereof. Which treatments are used depends on the type, location and grade of the cancer as well as the patient's health and preferences. In some embodiments, the therapy is chemotherapy or chemoradiation.

In one aspect, the disclosure features methods that include administering a therapeutically effective amount of a therapeutic agent to the subject in need thereof (e.g., a subject having, or identified or diagnosed as having, a cancer). In some embodiments, the subject has liver cancer (e.g., HCC).

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent is to be administered, a severity of symptoms and a route of administration, and thus administration can be determined on an individual basis.

In some embodiments, the methods described herein can be used to monitor the progression of the disease, determine the effectiveness of the treatment, and adjust treatment strategy. For example, cell free DNA can be collected from the subject to detect cancer and the information can also be used to select appropriate treatment for the subject. After the subject receives a treatment, cell free DNA can be collected from the subject. The analysis of these cfDNA can be used to monitor the progression of the disease, determine the effectiveness of the treatment, and/or adjust treatment strategy. In some embodiments, the results are then compared to the early results. In some embodiments, a dramatic decrease of HBV integration sites may suggest that the treatment is effective.

In some embodiments, the therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, carboplatin, nab-paclitaxel, paclitaxel, cisplatin, pemetrexed, gemcitabine, FOLFOX, or FOLFIRI are administered to the subject.

In some embodiments, the therapeutic agent is an antibody or antigen-binding fragment thereof. In some embodiments, the therapeutic agent is an antibody that specifically binds to PD-1, CTLA-4, BTLA, PD-L1, CD27, CD28, CD40, CD47, CD137, CD154, TIGIT, TIM-3, GITR, or OX40. In some embodiments, the therapeutic agent is an anti-PD-1 antibody, an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Systems, Software, and Interfaces

The methods described herein (e.g., quantifying, mapping, normalizing, range setting, adjusting, categorizing, counting and/or determining sequence reads, and counts) often require a computer, processor, software, module or other apparatus. Methods described herein typically are computer-implemented methods, and one or more portions of a method sometimes are performed by one or more processors. Embodiments pertaining to methods described herein generally are applicable to the same or related processes implemented by instructions in systems, apparatus and computer program products described herein. In some embodiments, processes and methods described herein are performed by automated methods. In some embodiments, an automated method is embodied in software, modules, processors, peripherals and/or an apparatus comprising the like, that determine sequence reads, counts, mapping, mapped sequence tags, elevations, profiles, normalizations, comparisons, range setting, categorization, adjustments, plotting, outcomes, transformations and identifications. As used herein, software refers to computer readable program instructions that, when executed by a processor, perform computer operations, as described herein.

Sequence reads, counts, elevations, and profiles derived from a subject (e.g., a control subject, a patient or a subject is suspected to have liver cancer) can be analyzed and processed to determine the presence or absence of a genetic variation (e.g., HBV integration sites). Sequence reads and counts sometimes are referred to as “data” or “datasets”. In some embodiments, data or datasets can be characterized by one or more features or variables. In some embodiments, the sequencing apparatus is included as part of the system. In some embodiments, a system comprises a computing apparatus and a sequencing apparatus, where the sequencing apparatus is configured to receive physical nucleic acid and generate sequence reads, and the computing apparatus is configured to process the reads from the sequencing apparatus. The computing apparatus sometimes is configured to determine the presence or absence of a genetic variation (e.g., HBV integration sites) from the sequence reads.

Implementations of the subject matter and the functional operations described herein can be implemented in digital electronic circuitry, in tangibly-embodied computer software or firmware, in computer hardware, including the structures described herein and their structural equivalents, or in combinations of one or more of the structures. Implementations of the subject matter described herein can be implemented as one or more computer programs, i.e., one or more modules of computer program instructions encoded on a tangible program carrier for execution by, or to control the operation of, a processing device. Alternatively, or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

Computers suitable for the execution of a computer program include, by way of example, general or special purpose microprocessors, or both, or any other kind of central processing unit. Generally, a central processing unit will receive instructions and information from a read only memory or a random access memory or both. The essential elements of a computer are a central processing unit for performing or executing instructions and one or more memory devices for storing instructions and information. Generally, a computer will also include, or be operatively coupled to receive information from or transfer information to, or both, one or more mass storage devices for storing information, e.g., magnetic, magneto optical disks, or optical disks. However, a computer need not have such devices.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

In one aspect, the disclosure provides a computer-implemented method for processing data in one or more data processing devices to process data as described herein, e.g., align sequence reads, map sequence reads to human genome or HBV genome, detect HBV integration sites, and/or determine whether a subject is likely to have HCC. In some embodiments, the computer-implemented method can output information indicative of the alignment, sequence mapping results, HBV integration sites, and/or the likelihood that the subject is likely to have HCC. In some embodiments, the disclosure provides one or more machine-readable hardware storage devices for processing data based on the methods as described herein. In some embodiments, the disclosure provides a system comprising one or more data processing devices; and one or more machine-readable hardware storage devices for processing data based on the methods as described herein.

Various types of mathematical models may be used to determine whether a subject has HCC, including, e.g., the regression model in the form of logistic regression, principal component analysis, linear discriminant analysis, correlated component analysis, etc. These models can be used in connection with data from different sets of sequencing results. The model for a given set of sequencing results is applied to a training dataset, generating relevant parameters for a classifier. In some cases, these models with relevant parameters for a classifier can be applied back to the training dataset, or applied to a validation (or test) dataset to evaluate the classifier. In some embodiments, the computer-implemented method includes the steps of inputting, into a classifier (e.g., a mathematical model), data representing one or more values for a classifier parameter that represents sequencing results (e.g., HBV integration sites, PE supporting reads, and splice supporting reads) from a test subject, with the classifier being for determining a likelihood score indicating whether the sequencing results classifies with (A) a set of sequencing results for a first group of individuals with HCC; as opposed to classifying with (B) a set of sequencing results for a second group of individuals without HCC; for each of one or more of the sequencing results, binding, by the one or more data processing devices, to the classifier parameter one or more values representing sequencing results; applying, by the one or more data processing devices, the classifier to bound values for the parameter; determining, by the one or more data processing devices based on application of the classifier, the likelihood score for the subject has HCC.

### Kits

The present disclosure also provides kits for collecting, transporting, and/or analyzing samples. Such a kit can include materials and reagents required for obtaining an appropriate sample (e.g., cfDNA or ctDNA) from a subject. In some embodiments, the kits include those materials and reagents that would be required for obtaining and storing a sample from a subject. The sample is then shipped to a service center for further processing (e.g., sequencing and/or data analysis).

The kits may further include instructions for collect the samples, performing the assay and methods for interpreting and analyzing the data resulting from the performance of the assay.

### EXAMPLES

The invention is further described in the following examples, which do not limit the scope of the invention described in the claims.

### Example 1: Sample Preparation

Samples were collected from several hepatocellular carcinoma patients for analysis.

### 1. Subjects

Five liver cancer patients were selected. All patients had also been diagnosed with concurrent HBV infection. One healthy person who has never been infected by HBV was selected as a negative control.

TABLE 1
Demographic Characteristics of the Subjects

| Sample | | | | Clinical |
|---|---|---|---|---|
| Number | Age | Gender | disease | Stages |
| #1 | 79 | male | Liver Cancer | III |
| #2 | 43 | male | Liver Cancer | III |
| #3 | 46 | male | Liver Cancer | III |
| #4 | 55 | male | Liver Cancer | I |
| #5 | 34 | male | Liver Cancer | I |
| #N01 | 54 | male | Normal | Negative |
| | | | | control |

### 2. Plasma Isolation and DNA Extraction

The subjects' blood samples were collected in tubes containing EDTA and centrifuged at 1600 g for 10 min at 4° C. within 2 hours of collection. The supernatants were further centrifuged at 16,000 g for 10 min at 4° C. Plasma was harvested and stored at −80° C. for further use. DNA from plasma was extracted from at least 2 mL plasma using the QIAamp Circulating Nucleic Acid kit (QIAGEN, Hilden, Germany) according to the manufacturers' instructions. DNA was quantified with the Qubit 4.0 Fluorometer and the Qubit dsDNA HS Assay kit (Life Technologies, Carlsbad, Calif.) according to the recommended protocol.

### 3. Library Construction

a) End Repair and A-Tailing

End repair was performed to ensure that DNA molecules were free of overhangs. In addition, for Illumina libraries and some libraries intended for the 454™ platform, A-tailing is usually required for incorporating of a non-template deoxyadenosine 5′-monophosphate (dAMP) onto the 3′ end of blunted DNA fragments. The end repair and A-tailing were prepared using KAPA Hyper Prep Kit (Kapa Biosystems, Wilmington, Mass.). The reactions were set up in a tube or well of a PCR plate. The conditions were shown in the table below:

TABLE 2

| Reagent | Volume | |
|---|---|---|
| cfDNA | 50 | μL |
| End Repair & A-Tailing Buffer | 7 | μL |
| End Repair & A-Tailing enzyme mix | 3 | μL |
| Total volume | 60 | μL |

TABLE 3

| | cfDNA | cfDNA | cfDNA |
|---|---|---|---|
| Sample | concentration | volume | quality |
| Number | (ng/μL) | (μL) | (ng) |
| #1 | 10.8 | 6.48 | 70 |
| #2 | 9.82 | 7.13 | 70 |
| #3 | 4.88 | 20.49 | 100 |
| #4 | 3.96 | 25.25 | 100 |
| #5 | 3.4 | 29.41 | 100 |
| #N01 | 0.606 | 50 | 30.3 |

The mixture was then incubated in a thermocycler programmed as outlined below:

TABLE 4

| Step | Temp | Time |
|---|---|---|
| End Repair and | 20° C. | 30 min |
| A-Tailing | 65° C. | 30 min |
| HOLD | 4° C. | ∞ |

b) Adapter Ligation

The adapter stocks were diluted to the appropriate concentration. In the same tube in which end repair and A-tailing was performed, adapter ligation reaction was performed with the following reagents.

TABLE 5

| Reagents | Volume | |
|---|---|---|
| End repair and A-tailing | 60 | μL |
| reaction product | | |
| PCR-grade water | 5 | μL |
| Ligation Buffer | 30 | μL |
| DNA Ligase | 10 | μL |
| Dilute adapter | 5 | μL |
| Total volume | 110 | μL |

The solutions were mixed thoroughly and centrifuged briefly, and then were incubated at 20° C. for 15 min.

The sequence of the adapters are shown below:

i7:
(SEQ ID NO: 1)
GATCGGAAGAGCACACGTCTGAACTCCAGTCACNNNNN
NNNATCTCGTATGCCGTCTTCTGCTTG
i5:
(SEQ ID NO: 2)
AATGATACGGCGACCACCGAGATCTACACNNNNNNNAC
ACTCTTTCCCTACACGACGCTCTTCCGATCT

c) Post-Ligation Cleanup

0.8× bead-based cleanup was performed by combining the following in a 1.5 ml tube:

TABLE 6

| Reagents | Volume | |
|---|---|---|
| Adapter ligation | 110 | μL |
| reaction product | | |
| Ampure XP beads | 88 | μL |
| Total volume | 198 | μL |

The reagents were mixed thoroughly and were incubated at room temperature for 15 min so that DNA can bind to the beads. The tube was then placed on a magnet to capture the beads. The supernatant was then discarded.

The tube was then kept on the magnet. 200 μL of 80% ethanol was added and incubated at room temperature for ≥30 seconds. The ethanol was then discarded. This process was repeated once. The beads were then dried at room temperature until all of the remaining ethanol evaporated. The beads were then thoroughly re-suspended in nuclease-free water.

The tube was incubated at room temperature for 5 min to elute DNA off the beads, and was then placed on a magnet to capture the beads. The supernatant was then transferred to a new tube and proceeded with library amplification.

d) Library Amplification

Library amplification reaction was performed with the following reagents.

TABLE 7

| Reagent | Volume | |
|---|---|---|
| 2 × KAPA HiFi Hotstart ReadyMix | 25 | μL |
| KAPA Library Amplification | 5 | μL |
| Primer Mix (10X) | | |
| Adapter-ligated library | 20 | μL |
| Total master mix volume | 50 | μL |

The reagents were then mixed thoroughly. Amplification was performed using the following protocol:

TABLE 8

| Step | Temp | Duration | | Cycles |
|---|---|---|---|---|
| Initial denaturation | 98° C. | 45 | s | 1 |
| Denaturation | 98° C. | 15 | s | Minimum number |
| Annealing* | 60° C. | 30 | s | required for optimal |
| Extension | 72° C. | 30 | s | Amplification as follow |
| Final extension | 72° C. | 1 | min | 1 |

| | | | |
|---|---|---|---|
| Hold | 4° C. | ∞ | 1 |

TABLE 9

| Sample Number | cycle |
|---|---|
| #1 | 7 |
| #2 | 7 |
| #3 | 4 |
| #4 | 4 |
| #5 | 4 |
| #N01 | 6 |

e) Post-Amplification Cleanup

1× bead-based cleanup was performed by combining the following reagents:

TABLE 10

| Reagent | Volume |
|---|---|
| Library amplification reaction product | 50 μL |
| Ampure XP beads | 50 μL |
| Total volume | 100 μL |

The reagents were mixed thoroughly and were incubated at room temperature for 15 min so that DNA can bind to the beads. The tube was then placed on a magnet to capture the beads. The supernatant was then discarded. 200 μL of 80% ethanol was then added. The tube was then incubated at room temperature for ≥30 sec. This procedure was repeated once. The beads were then dried at room temperature until all of the remaining ethanol evaporated. The beads were then thoroughly re-suspended in nuclease-free water.

The tube was then incubated at room temperature for 5 min to elute DNA off the beads, and was then placed on a magnet to capture the beads. The supernatant was then collected and transferred to a new tube.

TABLE 11

| | cfDNA library | cfDNA library | cfDNA library |
|---|---|---|---|
| Sample Number | concentration(ng/μL) | volume(μL) | quality(ng) |
| #1 | 84.8 | 21 | 1780.8 |
| #2 | 92.4 | 21 | 1940.4 |
| #3 | 28.8 | 35 | 1008 |
| #4 | 27 | 35 | 945 |
| #5 | 29.6 | 35 | 1036 |
| #N01 | 15.1 | 35 | 528.5 |

### 4. Hybridization

a) HBV Probe Hybridization

HBV probes (iGeneTech, Cat #AIHBC) were used to enrich cfDNA sequences that contain HBV sequences. The HBV probes are biotinylated oligonucleotides that cover the HBV genome.

The indexed cfDNA samples were pooled before hybridizing to the HBV probes. Each hybridization reaction requires a total of 750 ng indexed cfDNA.

For each capture reaction pool, indexed cfDNA library samples were combined with other reagents in one 0.2 mL PCR tube. Each final capture reaction pool should contain 750 ng indexed cfDNA. The reagents as shown in the table below were added. The PCR tube was labeled as “B tube.”

TABLE 12

| Reagent | Volume for 2 capture |
|---|---|
| Library | Total 750 ng |
| Hyb Human Block | 5 μL |
| Hyb Block-A0 | 1 μL |
| Hyb Index Block-8 | 5 μL |

TABLE 13

| Capture number | Sample Number | cfDNA library quality |
|---|---|---|
| B1 | #1 | 375 ng |
| | #2 | 375 ng |
| B2 | #3 | 375 ng |
| | #4 | 375 ng |
| B3 | #5 | 375 ng |
| | #N01 | 375 ng |

The volume in each tube was reduced to <10 μl by heating. Sufficient nuclease-free water was added to each concentrated cfDNA pool to bring the final well volume to 10 μl.

The tubes were then capped and spun in a centrifuge or mini-plate spinner to collect the liquid at the bottom of the wells. The wells were then placed in a thermal cycler.

TABLE 14

| Steps | Temperature | Time |
|---|---|---|
| Step1 | 95° C. | 5 min |
| Step2 | 65° C. | ∞ |

20 μL Hyb Buffer was added into a new 0.2 mL PCR tube. This tube was labeled as “A tube” and placed on the heating block.

5 μL RNase Block and 2 μL probe were added into a new 0.2 mL PCR tube and were mixed. The tube was labeled as “C tube”.

When the temperature of the thermal cycler drops to 65° C., 13 μL Hyb Buffer from A tube was transferred into the C tube. The reagents were mixed well and placed on thermal cycler. This tube was labeled as “AC tube”. After 2 min, the mix was transferred from AC tube into B tube, and then was mixed. The cap was closed and incubated at 65° C. for 16-24 h.

b) Streptavidin-Coated Magnetic Beads

Dynabeads MyOne Streptavidin T1 magnetic beads were suspended. For each hybridization sample, 50 μl of the resuspended beads were added to a new 1.5 mL tube. The tube was then placed in a magnetic separator device until the beads settled and the solution became clear. The supernatant was then discarded.

200 μl of Binding Buffer was added. The tube was then placed in a magnetic separator device until the beads settled and solution became clear. The supernatant was then discarded. This procedure was repeated 3 times.

The beads were resuspended in 200 μl of Binding Buffer. 200 μL of the washed beads were added to each well on a well plate for hybridization capture. Each hybridization mixture was transferred to the plate wells containing 200 μl of washed streptavidin beads and was fully mixed. The mixture was incubated on a Nutator mixer for 30 minutes at room temperature.

The beads were then collected and re-suspend in 200 μl of Wash buffer 1 (iGeneTech, Cat #TC2R-05). The wells were capped, placed on the capture place, and then incubated on a Nutator mixer for 15 minutes at room temperature. The plate was then placed in the magnetic separator until the solution was clear. The supernatant was discarded. The beads were then washed by Wash buffer 2 (iGeneTech, Cat #TC2R-05) three times. The supernatant was discarded.

The beads in each well were mixed with 30 μl of nuclease-free water on a vortex mixer for 5 seconds to resuspend the beads. The captured DNA was retained on the streptavidin beads during the post-capture amplification step.

Post-capture sample processing for multiplexed sequencing was performed. The appropriate volume of PCR reaction mixture was prepared. The samples were mixed using a vortex mixer and kept on ice.

TABLE 15

| Reagent | Volume |
|---|---|
| Captured DNA sample | 30 μL |
| Post PCR Buffer | 18 μL |
| Post PCR Primer(25 μM, for ILM) | 1 μL |
| Post PCR Polymerase | 1 μL |
| Total volume | 50 μL |

PCR was then performed.

TABLE 16

| Step | Temp | Duration | | Cycles |
|---|---|---|---|---|
| Initial denaturation | 95° C. | 4 | min | 1 |
| Denaturation | 98° C. | 20 | s | 15 |
| Annealing* | 60° C. | 30 | s | |
| Extension | 72° C. | 30 | s | |
| Final extension | 72° C. | 5 | min | 1 |

| | | | |
|---|---|---|---|
| Hold | 4° C. | ∞ | 1 |

The amplified captured libraries were purified using AMPure XP beads. 1× bead-based cleanup was performed.

TABLE 17

| Reagent | Volume |
|---|---|
| Library amplification reaction product | 55 μL |
| Ampure XP beads | 55 μL |
| Total volume | 110 μL |

The purified, amplified libraries were stored at −20° C. The library DNA was quantified with the Qubit 4.0 Fluorometer and the Qubit dsDNA HS Assay kit (Life Technologies) according to the recommended protocol.

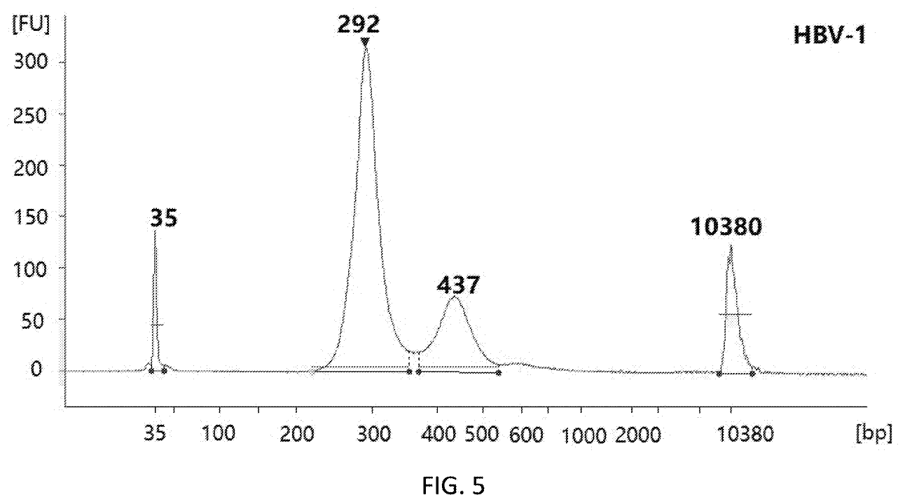

The fragment length was determined on a 2100 Bioanalyzer using the DNA 1000 Kit (Agilent). FIG. 5 shows the fragment lengths of DNA molecules in the HBV-1 library.

TABLE 18

| | | Post-PCR | | |
|---|---|---|---|---|
| Post-PCR | Sample | concentration | Post-PCR | Post-PCR |
| number | Number | (ng/μL) | volume(μL) | quality(ng) |
| HBV-1 | #1 | 13.3 | 25 | 332.5 |
| | #2 | | | |
| HBV-2 | #3 | 25 | 25 | 625 |
| | #4 | | | |
| HBV-3 | #5 | 39.4 | 25 | 985 |
| | #N01 | | | |

### Example 2: Data Processing

The purified, amplified libraries were sequenced by paired end sequencing. The analysis procedure was shown in FIG. 1. Filtering was performed. Reads with low quality or noise were removed. The following reads were removed:
(1) Reads with low quality (<20) rate more than 50%;
(2) The N content in one read is more than 5%;
(3) Cut the adapter sequence;

After filtering, the samples' sequence data was named as “clean data.” FastQC program was used to do quality control on the clean data (See e.g., Wingett, Steven W. et al. “FastQ Screen: A tool for multi-genome mapping and quality control.”

F1000Research 7 (2018)). If clean data passed Quality Control, BWA program was then used to align the human and HBV genome at same time (See e.g., Li et al., “Fast and accurate long-read alignment with Burrows-Wheeler transform.” Bioinformatics 26.5 (2010): 589-595). Then Samtools software was then used to sort mapping files by genome order, and mark duplication (See e.g., Li et al. “The sequence alignment/map format and SAMtools.” Bioinformatics 25.16 (2009): 2078-2079).

A method was developed to summarize the total mapping reads and the number of reads, which contains the HBV and human genome integration signals, as shown in FIGS. 2A-2B:
(1) “HBV mapping reads”: both of the paired end reads are mapped to the HBV genome. HBV mapping ratio (HBV mapping reads/Total reads)represent the virus content in the patient sample.
(2) “PE supporting reads”: One read of the paired end reads is mapped to the human genome and the other paired end read is mapped to the HBV genome (FIG. 2A).
(3) “Splicing supporting reads”: the integration site is located on at least one paired end read. Thus, a part of that paired end read is mapped to the human and a part of the same paired end read is mapped to the HBV genome (FIG. 2B).

“Splicing mapping reads” were extracted (FIG. 3 upper panel). For the sequencing reads, there might be duplicative sequences. The duplicative reads may be caused by natural sequence (the same DNA fragment has more than one copy in the sample, and thus is sequenced twice) or artificial sequence (during the sequencing process, a copy of the same sequence is created and sequenced). Samtools software was used to mark the duplicate reads. The number of different reads for item (1), (2), (3) were summarized. Both the total reads (including the duplicate reads) and the unique reads (excluding the duplicate reads) were summarized. The results were shown in the table below.

TABLE 19

| Sam- | | | | | | |
|---|---|---|---|---|---|---|
| ple | | | | | | |
| Name | #1 | #2 | #3 | #4 | #5 | #N01 |
| # of | 9321512 | 10297124 | 10674963 | 10791946 | 8129882 | 8669936 |
| total | | | | | | |
| clean | | | | | | |
| reads | | | | | | |
| # of | 18938 | 2303 | 71437 | 5156 | 934 | 0 |
| total | | | | | | |
| HBV | | | | | | |
| map- | | | | | | |
| ping | | | | | | |
| reads | | | | | | |
| # of | 11007 | 1444 | 38759 | 3587 | 720 | 0 |
| unique | | | | | | |
| HBV | | | | | | |
| map- | | | | | | |
| ping | | | | | | |
| PE | | | | | | |
| reads | | | | | | |
| # of | 4121 | 150 | 1371 | 44 | 21 | 0 |
| total | | | | | | |
| PE | | | | | | |
| sup- | | | | | | |
| port- | | | | | | |
| ing | | | | | | |
| reads | | | | | | |
| # of | 2113 | 93 | 967 | 40 | 19 | 0 |
| unique | | | | | | |
| PE | | | | | | |
| sup- | | | | | | |
| port- | | | | | | |
| ing | | | | | | |
| reads | | | | | | |
| # of | 163 | 7 | 205 | 7 | 3 | 0 |
| total | | | | | | |
| splic- | | | | | | |
| ing | | | | | | |
| sup- | | | | | | |
| port- | | | | | | |
| ing | | | | | | |
| Reads | | | | | | |
| # of | 99 | 5 | 143 | 5 | 2 | 0 |
| unique | | | | | | |
| splic- | | | | | | |
| ing | | | | | | |
| sup- | | | | | | |
| port- | | | | | | |
| ing | | | | | | |
| Reads | | | | | | |

Based on the genome mapping position of “splicing supporting reads”, the human genome and HBV genome breakpoints were calculated. Then extract 500 bp ‘fasta’ sequence near the breakpoints from human/HBV genome, joint the human and HBV ‘fasta’ sequence as integrating contig were rebuilt by BWA software.

An example of breakpoints of sample #3 was shown in the table below:

TABLE 20

| | | # of | |
|---|---|---|---|
| | | splicing | |
| | | support | |
| Breakpoints1 | Breakpoints2 | reads | ID of splicing support reads |
| chr16:11014376 | NC_003977.2:2408 | 20 | ST-E00244:687:H2MLYCCX2:7:1101:13190:44415, |
| | | | ST-E00244:687:H2MLYCCX2:7:1101:31832:40038, |
| | | | ST-E00244:687:H2MLYCCX2:7:1103:18873:44837, |
| | | | . . . |
| chr5:9747036 | NC_003977.2:465 | 51 | ST-E00244:687:H2MLYCCX2:7:1103:23480:20049, |
| | | | ST-E00244:687:H2MLYCCX2:7:1108:7882:13351, |
| | | | ST-E00244:687:H2MLYCCX2:7:1112:9678:61784, |
| | | | . . . |

When the integration breakpoints were identified, the integration site that were close to each other were merged. The length between the location of breakpoints1 was less than 50 bp, which was same to breakpoints2. If such the integration sites exist, the integration sites with less splicing support reads were removed. In the table below, the first integration event was removed.

TABLE 21

| Breakpoints1 | Breakpoints2 | # of splicing support reads |
|---|---|---|
| NC_003977.2:1442 | chr5:1250437 | 1 |
| NC_003977.2:1451 | chr5:1250436 | 92 |

Then, almost 500 bp around the breakpoints1 and breakpoints2 were joined to re-construct integration contig sequence, for example:

>NC_003977.2:951-1451:500 BP_chr5:1250436-
1250936:500 BP
(SEQ ID NO: 3)
AGAAAACTTCCTATTAACAGGCCTATTGATTGGAAAGTATGTCAACGA
ATTGTGGGTCTTTTGGGTTTTGCTGCCCCTTTTACACAATGTGGTTAT
CCTGCGTTGATGCCTTTGTATGCATGTATTCAATCTAAGCAGGCTTTC
ACTTTCTCGCCAACTTACAAGGCCTTTCTGTGTAAACAATACCTGAAC
CTTTACCCCGTTGCCCGGCAACGGCCAGGTCTGTGCCAAGTGTTTGCT
GACGCAACCCCCACTGGCTGGGGCTTGGTCATGGGCCATCAGCGCATG
CGTGGAACCTTTTCGGCTCCTCTGCCGATCCATACTGCGGAACTCCTA
GCCGCTTGTTTTGCTCGCAGCAGGTCTGGAGCAAACATTATCGGGACT
GATAACTCTGTTGTCCTATCCCGCAAATATACATCGTTTCCATGGCTG
CTAGGCTGTGCTGCCAACTGGATCCTGCGCGGGACGTCCTTTGTTTAC
GTCCCGTCGGCGCTGAATCCTgcctccctctctcacttctagggaccc
ttgtggccatatcaggcccaccagataatccaggatgaccttaagatc
ggctgactggcagccgtgattccacctgcagcctccaccagcctctgc
cttgcgggtgacacattcacaggttccaggagaaggacgtgggcatct
ttggggaggggctgtaattgtgcctgccacaAGTGCCTGGGGCTTCTG
AAACCCACCAAAGTTTGGCAAGCCCCCTGCACAGCATCCTTCCCAGGT
GGGCACCTGGCACCAACATCGACGGTTACAGCAGGTGCAGGACCGGCA
GGAGCGTGGGGCTGAGGCAGGAAAACAACCACTCCCTTTCAGGGGTCC
TGGCTGGTGTCACCCACAGCCTCCACCCTTGCCTGCTTCTCCTCCCTT
TCTGCTTTGAACTCACTCGCTCCATACACGCTTGTCTGTGGAAGGAAG
CTGCTTGAGATGAAGTTCAGGCCTAAGGAAGTCCAAAGAGCT

The BWA software was then used to build the mapping index. The PE supporting reads, Splicing supporting reads, Splicing mapping result were converted to “fatstq” format. The format was the same as the clean data format. The “fastq” file was aligned to the previous re-constructed integration contig sequence in order to obtain the mapping result (bam file).

Mapping reads (reads R1 and R2 from the same fragment were mapped on the same integration contig sequence, at an expected distance with the correct directions) with high mapping quality (>30) were extracted. The PE supporting reads and Splicing supporting reads for each reference integration contig were calculated. The results of sample #3 was shown in the table below as an example:

TABLE 22

| | # of Splicing_supporting | # of PE_supporting |
|---|---|---|
| Integration_Contig | reads | reads |
| chr5:9746536-9747036:500BP_NC_003977.2:465- | 695 | 6 |
| 965:500BP | | |
| NC_003977.2:951-1451:500BP_chr5:1250436- | 324 | 6 |
| 1250936:500BP | | |
| chr3:124667703-124668203:500BP_NC_003977.2:185- | 290 | 7 |
| 685:500BP | | |
| chr16:11013876-11014376:500BP_NC_003977.2:2408- | 147 | 0 |
| 2908:500BP | | |
| chr12:33110845-33111345:500BP_NC_003977.2:327- | 8 | 0 |
| 827:500BP | | |
| NC_003977.2:1258-1758:500BP_chr10:24370224- | 1 | 0 |
| 24370724:500BP | | |
| chr18:51598625-51599125:500BP_NC_003977.2:465- | 1 | 0 |
| 965:500BP | | |
| chr4:15739061-15739561:500BP_NC_003977.2:577- | 1 | 0 |
| 1077:500BP | | |

Because the sample was plasma sample, the insert size was 150-180 bp. This sample was sequenced by PE150, so the number of Splicing support reads was significant more than that of PE supporting reads. If the total reads of Splicing support read and PE supporting reads were no more than 3, the corresponding contig sequence was removed.

The breakpoints from confident integration contigs were then annotated by the UCSC human database.

TABLE 23

| Region | Gene | Chr. | Position | Sample | Integration_Contig |
|---|---|---|---|---|---|
| intergenic | SLC6A18(dist = 4132), | 5 | 1250436 | #3 | NC_003977.2:951- |
| | TERT(dist = 2851) | | | | 1451:500BP_chr5:1250436- |
| | | | | | 1250936:500BP |
| intergenic | MUC13(dist = 14608), | 3 | 124668203 | #3 | chr3:124667703- |
| | HEG1(dist = 16351) | | | | 124668203:500BP_NC_003977.2: |
| | | | | | 185-685:500BP |
| intergenic | PKP2(dist = 61565), | 12 | 33111345 | #3 | chr12:33110845-33111345: |
| | SYT10(dist = 417003) | | | | 500BP_NC_03977.2: |
| | | | | | 327-827:500BP |
| intronic | CIITA | 16 | 11014376 | #3 | chr16:11013876- |
| | | | | | 11014376:500BP_NC_003977.2: |
| | | | | | 2408-2908:500BP |
| ncRNA_intronic | LINC02112 | 5 | 9747036 | #3 | chr5:9746536-9747036: |
| | | | | | 500BP_NC_003977.2: |
| | | | | | 465-965:500BP |

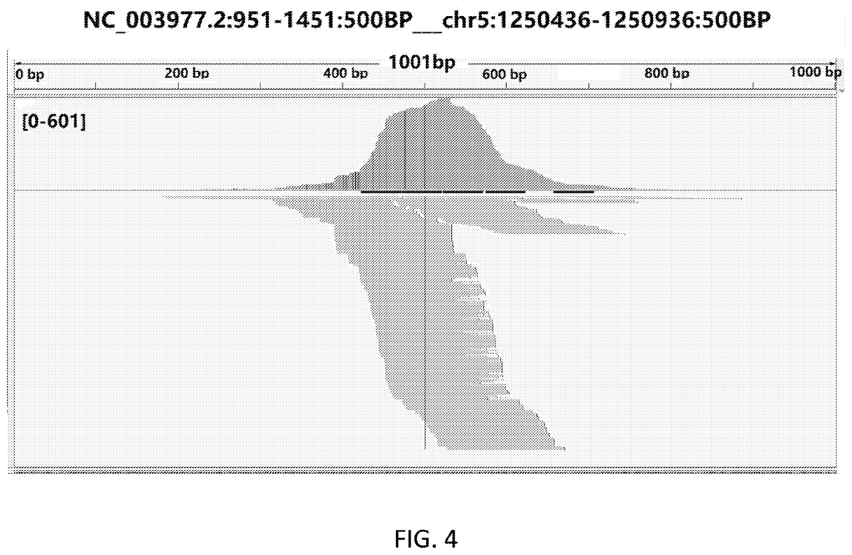

The first HBV integration contig from the table above was shown in FIG. 4. This integration site was also described in Sung, Wing-Kin, et al. “Genome-wide survey of recurrent HBV integration in hepatocellular carcinoma.” Nature genetics 44.7 (2012): 765, which confirms that the sequencing results are valid.

For the 6 test samples, each sample's integration sites were shown in the table below.

TABLE 24

| | | | | | | Splicing | PE |
|---|---|---|---|---|---|---|---|
| Region | Gene | Chr. | Pos. | Sample | Integration Contig. | supporting | supporting |
| intergenic | SLC6A18(dist = 4132), | 5 | 1250436 | #3 | NC_003977.2:951- | 324 | 6 |
| | TERT(dist = 2851) | | | | 1451:500BP_chr5: | | |
| | | | | | 1250436- | | |
| | | | | | 1250936:500BP | | |
| intergenic | MUC13(dist = 14608), | 3 | 124668203 | #3 | chr3:124667703- | 290 | 7 |
| | HEG1(dist = 16351) | | | | 124668203: | | |
| | | | | | 500BP_NC_003977.2: | | |
| | | | | | 185-685:500BP | | |
| intergenic | PKP2(dist = 61565), | 12 | 33111345 | #3 | chr12:33110845- | 8 | 0 |
| | SYT10(dist = 417003) | | | | 33111345: | | |
| | | | | | 500BP_NC_003977.2: | | |
| | | | | | 327-827:500BP | | |
| intronic | CIITA | 16 | 11014376 | #3 | chr16:11013876- | 147 | 0 |
| | | | | | 11014376: | | |
| | | | | | 500BP_NC_003977.2: | | |
| | | | | | 2408-2908:500BP | | |
| ncRNA_intronic | LINC02112 | 5 | 9747036 | #3 | chr5:9746536- | 695 | 6 |
| | | | | | 9747036: | | |
| | | | | | 500BP_NC_003977.2: | | |
| | | | | | 465-965:500BP | | |
| intergenic | LINC00351(dist = 16624), | 13 | 86135421 | #1 | chr13:86134921- | 36 | 0 |
| | SLITRK6(dist = 231501) | | | | 86135421: | | |
| | | | | | 500BP_NC_003977.2: | | |
| | | | | | 248-748:500BP | | |
| UTR5 | A4GALT(NM_001318038: | 22 | 43089958 | #1 | chr22:43089458- | 4 | 0 |
| | c.-1T > A, NM_017436: | | | | 43089958: | | |
| | c.-1T > A) | | | | 500BP_NC_003977.2: | | |
| | | | | | 1210-1710:500BP | | |
| ncRNA_intronic | LOC105378146 | 6 | 169357303 | #1 | chr6:169356803- | 6 | 0 |
| | | | | | 169357303: | | |
| | | | | | 500BP_NC_003977.2: | | |
| | | | | | 1195-1695:500BP | | |
| intergenic | CACNA2D1(dist = 112324), | 7 | 82185446 | #1 | chr7:82184946- | 4 | 0 |
| | PCLO(dist = 197875) | | | | 82185446: | | |
| | | | | | 500BP_NC_003977.2: | | |
| | | | | | 1212-1712:500BP | | |
| intergenic | CSMD3(dist = 376226), | 8 | 114825468 | #2 | NC_003977.2:1967- | 10 | 0 |
| | TRPS1(dist = 1595256) | | | | 2467:500BP_chr8: | | |
| | | | | | 114825468- | | |
| | | | | | 114825968:500BP | | |

Integration sites in Samples #1, #2 and #3 were detected with high confidence. Samples #4 and #5 contained integration supporting reads. The last sample #N01 (negative control) did not have integration signals.

### Example 3: Predicting subjects with HCC

A mathematical model is used to determine whether a subject has HCC. If the total reads of Splicing supporting read and PE supporting reads for a specific HBV integration site is greater than 3, this HBV integration site is confirmed with high confidence.

If one or more confirmed HBV integration sites are located in at least one of the HCC-related HBV integration genes, the subject is predicted to have HCC. The HCC-related HBV integration genes include e.g., TERT, MLL4, CCNE1, SENP5, ROCK1, FN1, PTPRD, UNC5D, NRG3, CTNND2, and AHRR.

Sample #3 one HBV integration site is located in a well-known TERT promoter region. Thus, Sample #3 is predicted as a HCC sample.

If the subject does not have any confirmed HBV integration sites that are located at HCC-related HBV integration genes, further analysis is required.

For further analysis, logistic regression is performed and applied to a dataset that includes a group of patients with HCC, and a group of patients without HCC. Samples are collected from these patients. The samples are processed by the methods as described in the early examples. The total number of unique PE supporting reads, the total number of unique splicing supporting reads, and the number of confirmed integration sites in subject are determined.

The total number of unique PE supporting reads, the total number of unique splicing supporting reads, and the number of confirmed integration sites are used as independent variables (“predictors”). The regression coefficients can be estimated using maximum likelihood estimation. The results can be used to determine the probability that a subject has HCC.

The probability that a sample has HCC can calculated based on the following equation:

$$P ( H C C ) = \frac{1}{1 + {e}^{- ( α + Σ {β}_{i} * {x}_{i} )}}$$

Wherein X₁ is the number of unique PE supporting reads, X₂ is the number of unique splicing supporting reads, and X₃ is the number of confirmed integration events.

If the coefficients from logistic regression are not readily available, the parameters based on the experience can be used:

α=−3; β₁=0.1; β₂=0.1; β₃=3

The X₁ value of samples #1, #2, #4, #5, #N01 are shown below, including the probability of predicting as HCC.

TABLE 25

| Sample Name | #1 | #2 | #4 | #5 | #N01 |
|---|---|---|---|---|---|
| # of unique PE supporting reads | 2113 | 93 | 40 | 19 | 0 |
| # of unique splicing supporting Reads | 99 | 5 | 5 | 2 | 0 |
| the number of confirmed integration events | 4 | 1 | 0 | 0 | 0 |
| Probability of being HCC | 1 | 1 | 0.82 | 0.29 | 0 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting an integration site of hepatitis B virus (HBV) viral DNA in the genome of a subject, the method comprising:
collecting a nucleic acid sample from the subject, wherein the nucleic acid sample is derived from whole blood or plasma of the subject;
enriching nucleic acids comprising HBV viral DNA sequences in the sample by hybridizing the nucleic acid sample to probes for HBV viral DNA;
sequencing the enriched nucleic acids, thereby obtaining a plurality of sequencing reads;
mapping the sequencing reads to both human genome and HBV genome; and
detecting the integration site of HBV viral DNA in the genome of the subject.

2-3. (canceled)

4. The method of claim 1, wherein the nucleic acid sample comprises cell free DNA (cfDNA).

5. The method of claim 1, wherein the nucleic acid sample comprises circulating tumor DNA (ctDNA).

6. The method of claim 1, wherein the probes for HBV viral DNA are prepared by amplifying or synthesizing HBV genomic DNA.

7. The method of claim 1, wherein the method further comprises:
identifying the subject as having hepatocellular carcinoma (HCC) if one or more integration sites for HBV viral DNA in the genome of the subject are detected.

8-10. (canceled)

11. The method of claim 7, wherein one or more integration sites are located in one or more HCC-related HBV integration genes selected from the group consisting of TERT, MLL4, CCNE1, SENP5, ROCK1, FN1, PTPRD, UNC5D, NRG3, CTNND2, AHRR, TERT, ABL1 (ABL), ABL2(ABLL,ARG), AKAP13 (HT31, LBC. BRX), ARAF1, ARHGEF5 (TIM), ATF1, AXL, BCL2, BRAF (BRAF1, RAFB1), BRCA1, BRCA2(FANCD1), BRIP1, CBL (CBL2), CSF1R (CSF-1, FMS, MCSF), DAPK1 (DAPK), DEK (D6S231E), DUSP6(MKP3,PYST1), EGF, EGFR (ERBB, ERBB1), ERBB3 (HER3), ERG; ETS1, ETS2, EWSR1 (EWS, ES, PNE,), FES (FPS), FGF4 (HSTF1, KFGF), FGFR1, FGFR10P (FOP), FLCN, FOS (c-fos), FUS (TLS), HRAS, GLI1, GLI2, GPC3, HER2 (ERBB2, TKR1, NEU), HGF (SF), IRF4 (LSIRF, MUM1), JUNB, KIT(SCFR), KRAS2 (RASK2), LCK, LCO, MAP3K8(TPL2, COT, EST), MCF2 (DBL), MDM2, MET(HGFR, RCCP2), MLH type genes, MMD, MOS (MSV), MRAS (RRAS3), MSH type genes, MYB (AMV), MYC, MYCL1 (LMYC), MYCN, NCOA4 (ELE1, ARA70, PTC3), NF1 type genes, NMYC, NRAS, NTRK1 (TRK, TRKA), NUP214 (CAN, D9S46E), OVC, TP53 (P53), PALB2, PAX3 (HUP2) STAT1, PDGFB (SIS), PIM genes, PML (MYL), PMS (PMSL) genes, PPM1D (WIP1), PTEN (MMAC1), PVT1, RAF1 (CRAF), RB1 (RB), RET, RRAS2 (TC21), ROS1 (ROS, MCF3), SMAD type genes, SMARCB1(SNF5, INI1), SMURF1, SRC (AVS), STAT1, STAT3, STATS, TDGF1 (CRGF), TGFBR2, THRA (ERBA, EAR7), TFG (TRKT3), TIF1 (TRIM24, TIF1A), TNC (TN, HXB), TRK, TUSC3, USP6 (TRE2), WNT1 (INT1), WT1, VHL, APC, CAPG, CDKN1A (CIP1, WAF1, p21), CDKN2A (CDKN2, MTS1(depreciated), TP16, p16(INK4)), CD99 (MIC2, MIC2X), FRAP1 (FRAP, MTOR, RAFT1), NF1, NF2, PI5, PDGFRL (PRLTS, PDGRL), PPARG PRKAR1A (TSE1), PRSS11 (HTRA, HTRA1)), RRAS, SEMA3B, SMAD2 (MADH2, MADR2), SMAD3 (MADH3), SMAD4 (MADH4, DPC4), ST3 (TSHL, CCTS), TET2, TOP1, TP63 (TP73L), TP73, TSG11, TUSC2 (FUS1), CD55, ICAM, MCAM, and ALCAM.

12. The method of claim 1, wherein the method further comprises:
identifying the subject as having hepatocellular carcinoma (HCC) if the total number of the integration sites for HBV viral DNA in the genome of the subject is over a reference threshold.

13. The method of claim 1, wherein the subject has hepatitis B.

14. The method of claim 7, wherein the method further comprises treating HCC in the subject.

15. A method of detecting an integration site of hepatitis B virus (HBV) viral DNA in the genome of a subject, the method comprising:
collecting a nucleic acid sample from the subject, wherein the nucleic acid sample is derived from whole blood or plasma of the subject;
sequencing the nucleic acid sample by paired end sequencing, thereby obtaining a plurality of paired end sequencing reads;
identifying one or more paired end sequencing reads that are mapped to a HBV integration site, wherein
(1) one end of the paired end sequencing reads is mapped to HBV viral DNA, and the other end of the paired end sequencing reads is mapped to human genome; or
(2) one end of the paired end sequencing reads comprises a sequence that is mapped to HBV viral DNA, and a sequence that is mapped to human genome;
detecting the integration site of HBV viral DNA in the subject.

16. The method of claim 15, wherein the method further comprises
prior to sequencing the nucleic acid sample by paired end sequencing, enriching nucleic acids comprising HBV sequences in the sample by hybridizing the nucleic acid sample to probes for HBV viral DNA.

17. The method of claim 15, wherein
(1) the integration site of HBV viral DNA has more than three paired end sequencing reads that are mapped to the HBV integration site; or
(2) the method further comprises constructing a HBV integration site sequence based on one or more paired end sequencing reads that are mapped to the HBV integration site;
and aligning one or more paired end sequencing reads to the constructed HBV integration site sequence.

18. (canceled)

19. The method of claim 15, wherein the method further comprises
determining one or more HBV integration sites are located in one or more genes selected from the group consisting of TERT, MLL4, CCNE1, SENP5, ROCK1, FN1, PTPRD, UNC5D, NRG3, CTNND2 and AHRR; and
determining that the subject has HCC.

20. The method of claim 15, wherein the method further comprises:
determining a probability that the subject has HCC based on one or more of the following:
(1) total number of paired end sequencing reads in the subject, each having one end that is mapped to HBV viral DNA, and one end that is mapped to human genome;
(2) total number of paired end sequencing reads in the subject, each having one end that comprises a sequence that is mapped to HBV viral DNA, and a sequence that is mapped to human genome; and
(3) total number of HBV integration sites in the subject.

21. The method of claim 20, wherein the probability is calculated based on the following equation:
$$P = \frac{1}{1 + {e}^{- ( α + {β}_{1} * {x}_{1} + {β}_{2} * {x}_{2} + {β}_{3} * {x}_{3} )}}$$
wherein X₁ is the total number of paired end sequencing reads in the subject, each having one end that is mapped to HBV viral DNA, and one end that is mapped to human genome;
X₂ is the total number of paired end sequencing reads in the subject, each having one end that comprises a sequence that is mapped to HBV viral DNA, and a sequence that is mapped to human genome;
X₃ is the total number of HBV integration sites in the subject; and
α is a constant, β₁, β₂, and β₃ are coefficients of a logistic regression.

22. The method of claim 15, wherein the subject has hepatitis B.

23. A method of screening a subject for hepatocellular carcinoma (HCC), the method comprising:
collecting a nucleic acid sample from the subject, wherein the nucleic acid sample is derived from whole blood or plasma of the subject;
sequencing the nucleic acid sample, thereby obtaining a plurality of sequencing reads;
mapping the sequencing reads to both human genome and HBV genome; and
detecting one or more integration sites of HBV viral DNA in the subject's genome, thereby determining that the subject has HCC.

24. The method of claim 23, wherein the method further comprises
enriching nucleic acids comprising HBV viral DNA sequences in the nucleic acid sample by hybridizing the nucleic acid sample to probes for HBV viral DNA.

25. The method of claim 23, wherein the nucleic acid sample is sequenced by paired end sequencing.

26.-30. (canceled)
